(19) United States
(12) Patent Application Publication
(10) Pub. No.: US 2016/0271402 A1
(43) Pub. Date: Sep. 22, 2016

(54) Implantable Electromedical Device

(71) Applicant: BIOTRONIK SE & Co. KG, Berlin (DE)

(72) Inventors: Thomas Sontheimer, Rosstal (DE); Josef Teske, Hallstadt (DE); Michael Arnold, Erlangen (DE)

(21) Appl. No.: 15/058,522

(22) Filed: Mar. 2, 2016

Related U.S. Application Data
(60) Provisional application No. 62/135,714, filed on Mar. 20, 2015.

(51) Int. Cl. A61N 1/375

(52) U.S. Cl. CPC A61N 1/3754

(57) ABSTRACT

An implantable electromedical device, including a device housing in which electronic and electrical function units are housed, a device head having at least one electrode or one line terminal, and a feedthrough arranged between the device housing and device head for at least one electrical conductor element connecting the electrodes or the line terminal to a function unit, wherein the feedthrough includes a one-piece plastic main body.

### CROSS-REFERENCE TO RELATED APPLICATIONS

This patent application claims the benefit of co-pending U.S. Provisional Patent Application No. 62/135,714, filed on Mar. 20, 2015, which is hereby incorporated by reference in its entirety.

### TECHNICAL FIELD

The present invention relates to an implantable electromedical device, comprising a device housing, in which electronic and electrical function units are housed, a device head having at least one electrode or a line terminal, and a feedthrough arranged between the device housing and device head for at least one electrical conductor element connecting the electrodes or the line terminal to a function unit.

### BACKGROUND

Such devices have long been used on a large scale as, for example, cardiac pacemakers or implantable cardioverters (especially defibrillators). This may, however, also be a less complex device, such as, for example, an electrode line or sensor line or even a cochlea implant.

Most implantable electromedical devices of practical significance are intended to deliver electrical pulses to excitable body tissue via suitably placed electrodes. In order to perform this function, electronic/electrical function units for generating the pulse and for suitably controlling the pulse generation are housed in the housing of the device, and electrodes or terminals for at least one electrode line are provided directly on the device externally, the electrodes being attached in the distal end portion thereof to the tissue for pulse transmission. The electronic/electrical function units in the device interior are to be connected to the outer electrodes or electrode line terminals in such a way that ensures utterly and permanently reliable function under the special conditions of the implanted state.

In particular, feedthroughs of which the main, insulating body consists substantially of ceramic or glass are known, wherein multilayer or multi-part superstructures have also been developed with use of metals or metal oxides and are used. Such known feedthroughs largely satisfy the requirements placed thereon. However, the thermal coefficients of expansion have to be taken into consideration when selecting the material for the components constituted by insulation ceramic/glass, metal solder or glass solder, metal pin and metal flange in order to be able to ensure a seal that is sufficient over the intended service life.

In the case of the conventional design (e.g., metal flange—solder—insulation ceramic—solder—metal pin), the effect of inappropriate coefficients of thermal expansion is evident primarily when cooling from the soldering temperature and when welding the feedthrough into the housing. This may result in mechanical tensile stresses, which may lead to material separation and, consequently, to potential leaks of the feedthrough. The ceramic and metal components used in conventional feedthroughs are interconnected by the solder material; with irregular expansion/shrinkage of the components, inclusive of the solder, due to heating/cooling processes, the resultant relative changes in length produce corresponding mechanical stresses, until the strength values of the used materials are opposed to a further rise in elastic stress. Ductile components/materials (for example, a flange made of titanium or a gold solder) reach the yield point thereof and convert a further change in length into a plastic deformation with a moderate rise in stress. Brittle components/materials (for example, an insulator made of Al₂O₃ ceramic), but also brittle phases produced during soldering (for example, between gold solder and titanium) by contrast reach the tensile strength thereof by the early occurrence of a material separation, which entails crack formation and possibly a leakage of the feedthrough.

Furthermore, the materials used conventionally are very costly materials, which then, in turn, require very costly joining processes, such as, for example, coating and high-temperature soldering.

European Patent No. EP 2 232 646 discloses a hermetically tight feedthrough structure that comprises a multi-part main or insulating body in combination with sealing (not structure-bearing) polymer layers. The production of such a feedthrough is highly complex in terms of the necessary process and test steps and also in terms of the pre-fabrication, storage and supply of many different parts.

U.S. Pat. No. 7,064,270 also describes a feedthrough formed in a number of parts that has been developed specifically for an electrode line and may comprise a number of components manufactured from plastic or provided with a plastic coating.

European Patent Application No. EP 2 388 044 discloses an electronic device that has a feedthrough that is of simple structure in principle and that is made of a liquid-crystalline polymer. Details concerning the device construction are not disclosed in this document.

The present invention is directed toward overcoming one or more of the above-mentioned problems.

### SUMMARY

An object of the present invention is to provide an improved implantable electromedical device that can be produced economically and that is reliable to a high degree.

At least this object is achieved by a device having the features of claim 1. Expedient developments of the inventive concept are specified in the dependent claims.

The present invention is based on the consideration of developing a feedthrough in which a material that tolerates the different coefficients of thermal expansion of the used components, has a low gas and liquid diffusion between each side of the feedthrough, and additionally also enables economical production, is used as joining component between the metal conductors and the flange. It includes the notion of providing a one-piece plastic main body for this purpose. This performs both the function of the supporting part and also the function of holding together the conductor elements or of holding together the conductor element or the conductor elements and a surrounding feedthrough flange, that is to say the function of electrically insulating these parts reliably with respect to one another. The extremely simple structure and the easy and cost-effective production and also the low material costs are advantageous here.

In the design phase, it is therefore no longer necessary to match the materials used in terms of the coefficients of thermal expansion thereof. It is thus made possible to use or to include advanced designs/materials that increase the degrees of freedom for the design of the implant housing.

The use of a polymer for the feedthrough main body and simultaneously as an insulator also provides for one or more of the following advantages:
A thermoset material can be introduced and cured at room temperature, such that there is no thermal loading, as is produced when soldering a conventional feedthrough.
Thermal stresses that occur when a hot polymer melt made of a thermoplastic is introduced and cooled and when the flange is welded into a housing are lower than with conventionally produced feedthroughs, as expected, since the polymer material has a much lower modulus of elasticity compared to metal materials.

In addition, completely new cost-saving potentials are enabled; however, injection molding is the method to be used to produce components at high speed, in high numbers and at low cost.

The production process is particularly simple and economical in an embodiment of the plastic main body as an injection molded part. In a variant of this embodiment, the plastic main body is formed by injecting a surrounding separate feedthrough flange and by encapsulating at least one terminal pin, in particular, a number of terminal pins, by means of injection molding.

In further embodiments, the plastic main body has a filling with non-organic and non-metal particles, in particular, glass and/or ceramic particles. In particular, the particles for filling the plastic main body may have a mean particle size of less than 20 μm, and in particular of less than 10 μm. In order to meet special requirements, other particle sizes can also be considered, and the degree of filling of the plastic with the additive can be set in view of the special physical requirements and relevant properties of the plastic and additive used.

In embodiments of practical relevance, the plastic main body is formed with a thermoplastic or thermoset plastic, in particular, an epoxy resin, polysulfone, PEEK or a liquid-crystalline polymer. Plastics other than those mentioned here can also be considered.

In further embodiments, at least one extension extending into the plastic main body, in order to lengthen a diffusion path extending from the surface of the plastic main body outside the housing to the surface of the plastic main body inside the housing, is provided on the device housing and/or the separate feedthrough flange and/or at least one electrical conductor element. In a special embodiment, the separate feedthrough flange has a number of extensions extending substantially perpendicularly to the peripheral surface of said flange and/or the, or each, injected terminal pin has at least one disc-shaped extension extending perpendicularly to the longitudinal extent of said pin(s). It goes without saying that the extensions on the individual parts are arranged relative to one another in such a way that they do not contact one another, but have distances from one another sufficient for effective electrical insulation.

In a further embodiment, the separate feedthrough flange has an inserted ground terminal.

In a further embodiment, the separate feedthrough flange is embodied as a side part with an injected ground terminal formed by metal injection molding. This is an established method for economical production of high-quality metal parts with which a person skilled in the art is familiar per se and which therefore does not have to be described in greater detail. In particular, titanium or a titanium alloy is considered as a metal used for carrying out the present invention; in principle, however, related metals such as, for example, niobium, molybdenum, tantalum, tungsten, vanadium, zirconium or iridium and alloys thereof or nickel or palladium or alloys thereof or steels, in particular medical steels, such as, for example, 316L, can be used.

In further embodiments of the present invention, one or more barrier layer(s), which in particular is/are biocompatible, is/are applied to the surface of the plastic main body outside and/or inside the housing, said barrier layer(s) extending over the respective total surface and preferably also the adjacent region of the inner periphery of the feedthrough flange. Such a barrier made of one or more thin layers improves the tightness of the feedthrough of the device, in particular, in terms of the gas and liquid diffusion from surrounding bodily fluid into the device. The biocompatibility of the coating(s) is of importance primarily in an application outside the device; with provision of a two-sided coating, however, it is advantageous to form this from the same layer material.

In a possible variant of the above-mentioned embodiment, the barrier layer/the barrier layers is/are formed as thin layer(s) applied by vacuum coating. In particular, the barrier layer or the layer system has a metal oxide layer, in particular, titanium oxide, aluminum oxide, silicon oxide, niobium oxide, or the like. The production of such metal oxide layers is easily possible with known coating methods and economically commercially available targets and is well known to a person skilled and, therefore, this variant does not require any further description.

The conductor elements of the implant feedthrough can consist, for example, of the elements Pt, Ir, Nb, Ta, Ti, Fe, Cr, Ni, or alloys thereof.

In a further embodiment, the conductor elements can be formed in two parts, for example, each having a soft-solderable region on the implant inner side, for example, made of Cu, Ni, Au, Ag, or alloys thereof, and each having a weldable, biocompatible region on the implant outer side, for example, made of Ti, Nb, Ta, Fe, Ni, Cr, or alloys thereof. This embodiment has the advantage that it can also be fixedly attached electrically and mechanically to an electronic module in what is known as a reflow soldering process, similarly to any other surface-mountable (“SMT”) electronic component, without having to provide bores for the terminal pins in the electronic module. These two-part pins advantageously can be produced economically in the following process sequence: rolling, punching, and forming, with possible intermediate annealing steps. The ground conductor element can also be produced in this way, not just the other conductor elements that are responsible for the signal transmission.

In a further embodiment, a high-frequency filter can be joined directly to the signal-transmitting conductor elements, for example, by soft soldering, welding, conductive bonding or clamping. This high-frequency filter is embodied as a low-pass filter or as a band-stop and has the task of holding back possible high-frequency interfering radiation from the implant housing interior. In a further advantageous embodiment, the high-frequency filter can be fixedly cast in mechanically, directly from the plastic body of the feedthrough.

Further embodiments, features, aspects, objects, advantages, and possible applications of the present invention could be learned from the following description, in combination with the Figures, and the appended claims.

### DESCRIPTION OF THE DRAWINGS

Advantages and expedient features of the present invention will also emerge from the description of exemplary embodiments with reference to the Figures, in which:

FIG. 1 shows a schematic, partly cut illustration of an implantable electromedical device.

FIG. 2 shows a schematic cross-sectional illustration (partial view) of an exemplary embodiment of the present invention.

FIG. 3 shows a schematic cross-sectional illustration (partial view) of a further exemplary embodiment of the present invention.

### DETAILED DESCRIPTION

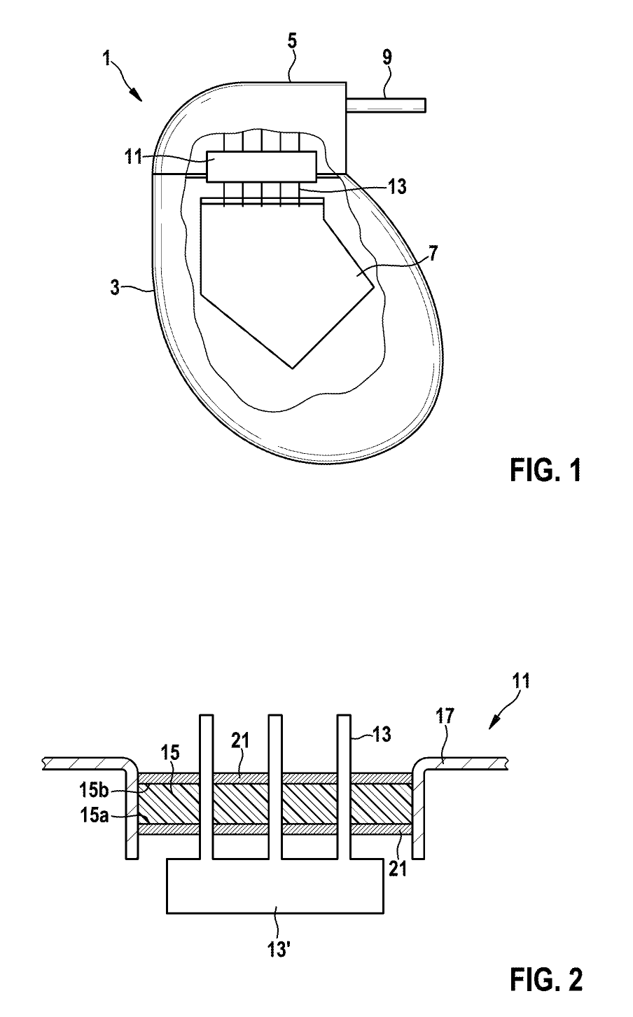

FIG. 1 shows a cardiac pacemaker 1 with a pacemaker housing 3 and a head part (header) 5, in the interior of which a printed circuit board (“PCB”) 7 is arranged in addition to other electronic components, an electrode line 9 being connected to the line terminal (not shown) of said printed circuit board 7, which line terminal is arranged in the header 5. A feedthrough 11 provided between the device housing 3 and header 5 comprises a plurality of terminal pins 13. The terminal pins 13 are plugged at one end through a corresponding bore in the printed circuit board 7 and are soft-soldered thereto.

FIG. 2, in the form of a cross-sectional illustration, schematically shows a first embodiment of the feedthrough 11, specifically with an injection-molded plastic main body 15 in a cold-formed feedthrough flange 17, wherein the plastic main body 15 is provided both on the surface 15a outside the device and on the surface 15b inside the device with a diffusion-inhibiting barrier layer system 21 (formed, for example, by sputtering or vacuum deposition). Terminal pins 13 are injected into the plastic main body 15 as conductor elements. In the illustration, a punched metal comb 13′ can be seen, which serves to fix the conductor elements 13 in the injection mold and is removed with manufacture of the feedthrough. It can be galvanized, for example, with Au, whereby Au-coated and therefore soft-solderable terminal pins are provided.

In further embodiments (without drawing), the plastic main body 15 is provided in each case only on one side (either on the side inside the device or on the side outside the device) with a diffusion-inhibiting barrier layer system 21.

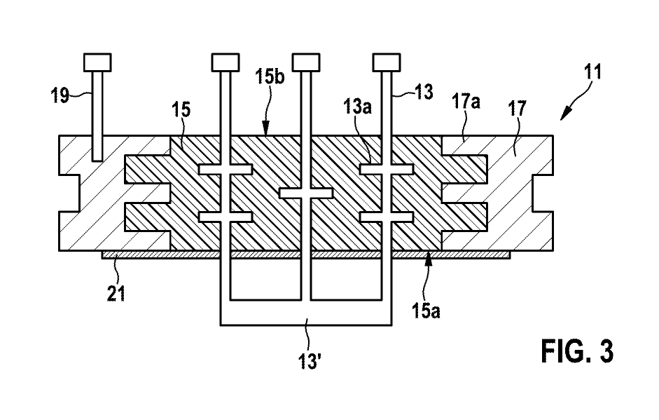

FIG. 3, in an illustration based on FIG. 2 and with use of the same reference numerals for functionally like parts, shows a modified embodiment of the feedthrough 11. In this case, a flange 17, pre-formed by metal injection molding (“MIM”) technology, is provided instead of a cold-formed flange and has, on the inner periphery thereof, a number of annular extensions 17a protruding inwardly into the material of the plastic main body 15. Correspondingly, the terminal pins 13, in the embodiment according to FIG. 3, are provided with one or two fixing discs 13a, which, similarly to the annular extensions 17a, also mesh with the polymer material of the main body 15 and at the same time protrude thereinto in an offset manner (e.g., in a 2-1-2-1-2-1 etc. manner), such that a lengthened diffusion path is created between the surface 15a outside the device and the surface 15b inside the device. In order to additionally improve the diffusion strength with respect to gaseous or liquid components of bodily fluid, which surrounds the electromedical device during use, a barrier layer system 21 is applied here to the side outside the device.

The embodiment(s) of the present invention is/are also possible in a large number of modifications of the shown examples and above-highlighted aspects of the present invention. In particular, the geometric design of the feedthrough flange (provided such a flange is provided separately) and of the conductor elements (e.g., terminal pins) can be modified in a number of ways, and flanges and/or terminal pins pre-fabricated in a different way can be embedded in the plastic main body of the feedthrough. A diffusion barrier layer system formed of one or more thin layers can be provided on both surfaces of the main body, or can also be completely omitted, depending on diffusion properties of the used plastic and of the optionally provided filling.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range.

I/WE claim:

1. An implantable electromedical device, comprising
a device housing in which electronic and electrical function units are housed;
a device head having at least one electrode and one line terminal; and
a feedthrough arranged between the device housing and the device head for at least one electrical conductor element connecting the electrodes or the line terminal to a function unit,
wherein the feedthrough comprises a one-piece plastic main body.

2. The device according to claim 1, wherein the plastic main body is formed as an injection-molded part.

3. The device according to claim 2, wherein the plastic main body is formed by injecting a surrounding separate feedthrough flange and encapsulating at least one terminal pin by means of injection molding.

4. The device according to claim 1, wherein the plastic main body has a filling with non-organic and non-metal particles including glass and/or ceramic particles.

5. The device according to claim 4, wherein the particles for filling the plastic main body have a mean particle size of less than 20 μm, in particular of less than 10 μm.

6. The device according to claim 4, wherein the particles for filling the plastic main body have a mean particle size of less than 10 μm.

7. The device according to claim 1, wherein the plastic main body is formed with a thermoplastic or thermoset plastic including an epoxy resin, polysulfone, PEEK or a liquid-crystalline polymer.

8. The device according to claim 3, wherein at least one extension extending into the plastic main body in order to lengthen a diffusion path extending from the surface of the plastic main body outside the housing to the surface of the plastic main body inside the housing is provided on the device housing and/or the separate feedthrough flange and/or at least one electrical conductor element.

9. The device according to claim 8, wherein the separate feedthrough flange has a plurality of extensions extending substantially perpendicularly to the peripheral surface thereof and/or the, or each, injected terminal pin has at least one disc-shaped extension extending perpendicularly to the longitudinal extent thereof.

10. The device according to claim 3, wherein the separate feedthrough flange has an inserted ground terminal.

11. The device according to claim 3, wherein the separate feedthrough flange is embodied as a side part with injected ground terminal formed by metal injection molding.

12. The device according to claim 3, wherein a barrier layer, which is biocompatible, is applied to the surface of the plastic main body outside the housing and/or to the surface of the plastic main body inside the housing, said barrier layer extending over the respective total surface and also the adjacent region of the inner periphery of the feedthrough flange.

13. The device according to claim 12, wherein a barrier layer system is provided with one or more thin layer(s) applied by vacuum coating(s).

14. The device according to claim 13, wherein the barrier layer or the barrier layer system has at least one metal oxide layer including titanium oxide, aluminum oxide, silicon oxide, niobium oxide, or the like.

15. The device according to claim 1, wherein the conductor element is formed as a substantially cylindrical terminal pin which has an end formed in the manner of a needle head.

16. The device according to claim 1, formed as an electrostimulation device including a cardiac pacemaker or cardioverter, wherein the line terminal is formed on the device head for connection of an electrode line.
